(19) United States
(12) Patent Application Publication
(10) Pub. No.: US 2026/0348932 A1
(43) Pub. Date: Nov. 5, 2026

(54) MOBILE PLATFORM AIR QUALITY SENSOR WITH ADAPTIVE AIRFLOW REGULATION STRUCTURE

(71) Applicant: The Regents of the University of California, Oakland, CA (US)

(72) Inventors: Shijia Pan, Merced, CA (US); Joshua Zhang, Merced, CA (US); Shubham Rohal, Merced, CA (US)

(21) Appl. No.: 19/655,208

(22) Filed: Apr. 22, 2026

Related U.S. Application Data
(60) Provisional application No. 63/792,605, filed on Apr. 22, 2025.

(51) Int. Cl. G01N 33/00

(52) U.S. Cl. CPC G01N 33/0009

(57) ABSTRACT

A mobile air quality monitoring system features an innovative airflow-regulating and energy-harvesting design for enhanced measurement accuracy and autonomy. The system includes an air quality sensor equipped with a flow adaptive assembly including a channel tube and an elastic flap. The channel tube stabilizes incoming airflow, while the elastic flap dynamically deforms in response to external airflow variations, ensuring consistent flow to the sensor. Designed for vehicle integration, the system utilizes vehicle-induced airflow, eliminating the need for traditional fans or pumps. A turbine mounted on the vehicle harvests kinetic energy from airflow, generating sufficient power to operate the sensor and an associated microcontroller unit (MCU). The MCU processes sensor data and transmits it to a computing device, which integrates additional information, such as location and speed, for calibration and real-time reporting. This system provides a cost-effective, energy-autonomous solution for fine-grained, mobile air quality monitoring.

### CROSS-REFERENCE TO RELATED APPLICATIONS

This application claims the benefit of, and priority to, U.S. Provisional Patent Application Ser. No. 63/792,605 filed on Apr. 22, 2025. The entire contents of the foregoing application are incorporated by reference herein.

### BACKGROUND

Fine-grained air quality monitoring is useful for safeguarding public health and addressing climate challenges. However, existing approaches to air quality measurements exhibit significant limitations. High-accuracy measurements from monitoring stations, while precise, provide limited spatial coverage, leaving large areas without adequate monitoring. Similarly, low-cost citizen science sensor networks often lack coverage.

Mobile platforms, including vehicles and drones, present a more efficient means of sampling air quality data due to their ability to cover broader geographic areas. Despite their potential, these solutions face challenges related to power requirements, complicated integration with mobile platforms, and prohibitively high costs, which collectively render them inaccessible to the general public.

Among various air pollutants, particulate matter with particles that are 2.5 micrometers or less in diameter (PM2.5) presents a particularly significant threat to public health. Exposure to PM2.5 is associated with severe health concerns, including stroke, asthma, and behavioral dysfunction. Current PM2.5 measurement methods primarily fall into three categories: (1) high-cost, high-accuracy systems such as microbalance-based PM monitoring stations, (2) low-cost citizen science sensor networks with uneven geographic distribution, and (3) mobile-based sensing solutions that leverage vehicles or drones to improve sampling coverage. Traditional low-cost air quality sensors are designed for stationary deployment, relying on a fan to draw airflow. When installed on mobile platforms, external airflow disrupts this mechanism, affecting sensor accuracy. To address this, certain mobile solutions utilize expensive housings with air pumps to regulate airflow. Integrating air quality sensors into vehicles also requires significant retrofitting, including wiring to the vehicle's power source, complicating scalability. The present disclosure overcomes these drawbacks of mobile air quality sensors. Thus, there is need for an accessible, cost-effective, and adaptable solution to enable comprehensive and fine-grained, mobile air quality monitoring.

### SUMMARY

According to one embodiment of the present disclosure, a system for mobile air quality monitoring is described. The system includes an air quality sensor having a housing and an inlet defined in the housing. The system also includes a flow adaptive assembly coupled to the inlet of the air quality sensor. The flow adaptive assembly includes a channel tube extending from the housing, the channel tube having a length sufficient to stabilize airflow entering the air quality sensor and an elastic flap positioned within the channel tube, where the elastic flap is configured to deform in response to variations in the airflow entering the channel tube, thereby regulating flowrate of the stabilized airflow into the air quality sensor. The system further includes a microcontroller unit operatively connected to the air quality sensor. The microcontroller unit is configured to process data generated by the air quality sensor.

Implementations of the above embodiment may include one or more of the following features. According to one aspect of the above embodiment, the air quality sensor may be configured to be mounted on a vehicle, which generates the airflow entering the air quality sensor while moving. The flow adaptive assembly may further include a funnel coupled to an intake of the channel tube. The system may include a turbine coupled to the microcontroller unit, the turbine may be also configured to be mounted on the vehicle and harvest kinetic energy from the airflow. The turbine generates electrical energy to power the microcontroller unit and the air quality sensor. The system may also include a computing device operatively connected to the microcontroller unit via a wireless communication interface. The computing device may be configured to receive data from the microcontroller unit, integrate the data with location and speed information of the vehicle, and calibrate air quality measurements from the air quality sensor. The air quality sensor may be configured to measure particulate matter concentration in the air. The elastic flap may include a mounting portion for securing the flap within the channel tube and a flexing portion configured to deform under airflow pressure. The channel tube may have a cross-sectional geometry selected from circular, rectangular, oval, or polygonal shape, and may be adapted to optimize airflow stabilization. The elastic flap may be configured to deform within a pre-defined range of airflow velocities. The elastic flap may be disposed at an initial angle relative to a wall of the channel tube, and may maintain the initial angle until the airflow exceeds a threshold velocity. The initial angle may be from about 30° to about 60°.

According to another embodiment of the present disclosure, an air quality sensor is described. The air quality sensor includes a housing having a sensor component for measuring air quality and an inlet. The air quality sensor also includes a flow adaptive assembly coupled to the inlet. The flow adaptive assembly includes a channel tube extending from the housing, the channel tube having a sufficient length to stabilize airflow entering the air quality sensor; and an elastic flap positioned within the channel tube, where the elastic flap is configured to deform in response to variations in the airflow entering the channel tube, thereby regulating flowrate of the stabilized airflow into the air quality sensor.

Implementations of the above embodiment may include one or more of the following features. According to one aspect of the above embodiment, the elastic flap may include a mounting portion for securing the flap within the channel tube and a flexing portion configured to deform under airflow pressure. The flow adaptive assembly may further include a funnel coupled to an intake of the channel tube. The channel tube may have a circular, rectangular, oval, or polygonal cross-section. The elastic flap may be configured to deform within a pre-defined range of airflow velocities. The elastic flap may be disposed at an initial angle relative to a wall of the channel tube, and the elastic flap may be configured to maintain the initial angle until the airflow exceeds a threshold velocity. The initial angle may be 30° to 60°. The sensor component may be configured to measure particulate matter concentration in the air. The sensor component may include a laser module and a photodetector for measuring concentration of particulate matter in the stabilized airflow. Particulate matter may be at least one of PM1.0, PM2.5, or PM10. The sensor component may include a gas sensor configured to measure concentration or detect at least one of carbon monoxide, carbon dioxide, nitrogen dioxide, sulfur dioxide, ozone, a volatile organic compound, hydrogen sulfide, ammonia, or radon.

### BRIEF DESCRIPTION OF DRAWINGS

Various embodiments of the present disclosure are described herein below with reference to the figures wherein:

FIG. 1 is a schematic diagram of an air quality monitoring sensor with a fan to maintain airflow;

FIG. 2 is a schematic diagram of an air quality monitoring sensor with a flow adaptive assembly according to an embodiment of the present disclosure;

FIG. 3 is a system for measuring particulate matter concentration using the flow adaptive assembly according to an embodiment of the present disclosure;

FIGS. 4A-C show a side, cross-sectional view, a front view, and an enlarged view of the flow adaptive assembly of FIG. 2, respectively;

FIG. 5 is a deformation model of a cantilever flap of the flow adaptive assembly of FIG. 2;

FIG. 6 is a schematic diagram illustrating Venturi effect within the flow adaptive assembly of FIG. 2 showing flow velocity changes within a channel of varying sizes of cross-sectional area;

FIG. 7 shows an airflow regulation curve and corresponding shapes and angles of the flap of the flow adaptive assembly of FIG. 2 according to an embodiment of the present disclosure;

FIG. 8 is a schematic diagram of a system for measuring particulate matter concentration using the flow adaptive assembly according to an embodiment of the present disclosure;

FIG. 9 is a diagram of a sensor for measuring particulate matter concentration using the flow adaptive assembly according to an embodiment of the present disclosure;

FIG. 10 is a side, cross-sectional view of a flow adaptive assembly according to another embodiment of the present disclosure;

FIG. 11 is a side, cross-sectional view of a channel of the flow adaptive assembly of FIG. 10;

FIG. 12 is a deformation model of a cantilever flap of the flow adaptive assembly of FIG. 10;

FIG. 13 is a side, cross-sectional view of a funnel of the flow adaptive assembly of FIG. 10;

FIG. 14 shows a schematic diagram of simulated airflow paths through the flow adaptive assembly of FIG. 2 according to an embodiment of the present disclosure;

FIG. 15 shows a simulation heatmap of airflow velocity through the flow adaptive assembly of FIG. 2 according to an embodiment of the present disclosure;

FIG. 16 is an airflow regulation curve of flowrate as a function of external air speed and corresponding shape and angle of the flap of the flow adaptive assembly of FIG. 2;

FIG. 17 is a photograph of a wind speed sensor disposed on an automobile used for evaluating airflow speed on a moving vehicle;

FIG. 18 is a dot and fitted plot of wind speed as a function of vehicle speed obtained using the setup of FIG. 17;

FIG. 19 is an airflow regulation curve of flowrate as a function of external air speed with varying flap thickness;

FIG. 20 is an airflow regulation curve of flowrate as a function of external air speed with varying flap angle;

FIG. 21 is an airflow regulation curve of flowrate as a function of external air speed with varying flap length;

FIG. 22 shows power consumption plots by the air quality monitoring sensor with and without fans;

FIG. 23 is a power generation plot as a function of air velocity;

FIG. 24 is an airflow regulation curve of flowrate as a function of external air speed and corresponding shape and angle of the flap of the flow adaptive assembly of FIG. 10; and

FIG. 25 is an airflow regulation curve of flowrate as a function of external air speed and corresponding shape and angle of the flap of the flow adaptive assembly of FIG. 10.

### DETAILED DESCRIPTION

The present disclosure is related to mobile-based air quality monitoring using a novel sensor design. A plurality of the air quality sensors may be used to provide for broad spatial coverage. In particular, the sensors may be incorporated or disposed on vehicles to enable crowdsensing air quality. As vehicles move through a geographic area, air quality data is collected from the sensors on the vehicles and analyzed along the travel paths of each of the vehicles. The present disclosure overcomes the challenges that previously hindered adoption of mobile-based air quality sensing caused by vehicle movement-induced irregular airflow within air quality sensors, which impacts sensing precision.

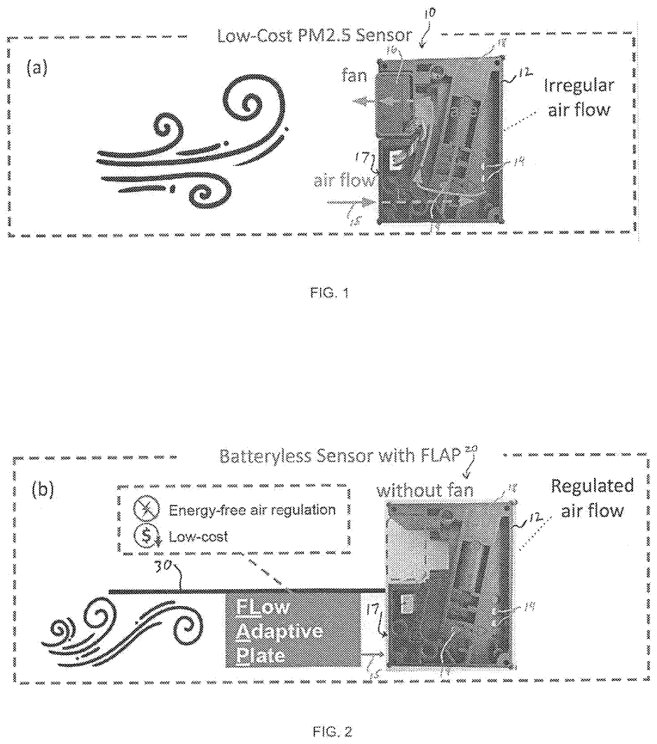

With reference to FIG. 1, an air quality monitoring sensor 10 is shown, which may include any suitable sensor components such as a particulate matter sensor configured to measure airborne particles of different sizes, such as PM1.0, PM2.5, and/or PM10. In embodiments, the sensor 10 may include a gas sensor for measuring concentration or detecting various compounds such as carbon monoxide, carbon dioxide, nitrogen dioxide, sulfur dioxide, ozone, volatile organic compounds, hydrogen sulfide, ammonia, radon, etc. In further embodiments, the sensor 10 may be also configured to measure temperature, humidity, and other parameters. Furthermore, the sensor may be a multi-functional sensor configured to measure or detect any one or more air properties or constituents.

The sensor 10 includes a housing 12 which secures and aligns various components, while preventing dust and external interference from contaminating the measurements. The housing 12 includes one or more channels 14, which guide airflow 15 through the sensor 10, ensuring the sensor components, e.g., laser module 18 and photodetector 19, interact with a representative sample. An air intake fan 16 is in fluid communication with the channels 14 to provide for airflow 15 that carries air through the sensor 10. The airflow enters the housing 12 through an inlet 17. This ensures a consistent airflow for measurement. The laser module 18 emits a light beam through the airflow 15. The laser is used in detecting particulate matter as light scatters off particles in the airflow 15. The photodetector 19 is disposed near laser module 18 and is positioned to capture scattered light from the particles. The photodetector 19 converts light intensity into electrical signals proportional to the concentration of particles. The sensor 10 also includes any other suitable electronics and circuitry needed for power and signal processing.

The present disclosure provides a solution to irregular airflow issues and simultaneously addresses the integration challenges of the sensor with vehicles by leveraging energy from airflow to power the sensor. Consequently, the proposed solution manipulates vehicle-induced airflow to (1) regulate airflow to replace fans or air pumps and (2) harvest energy for powering the sensor and/or controller coupled thereto. This disclosure provides a structural and mechanical solution for battery-free air quality sensing. As used herein, the term “battery-free” denotes energy harvesting, e.g., capturing airflow via a turbine that converts mechanical energy into electrical energy, radio frequency energy harvesting (RFEH) that converts electromagnetic (EM) energy from the environment into electrical energy, etc. The disclosure provides a flow adaptive assembly for regulating flowrate of the air flow using a self-adaptive flap that is integrated in front of the air quality sensor's airflow input. When the vehicle operates within a specific speed range, the structure adapts to varying external airflow velocities to maintain consistent airflow input to the sensor. Adjustable structural parameters allow the vehicle to operate in a range of speeds to achieve effective airflow regulation.

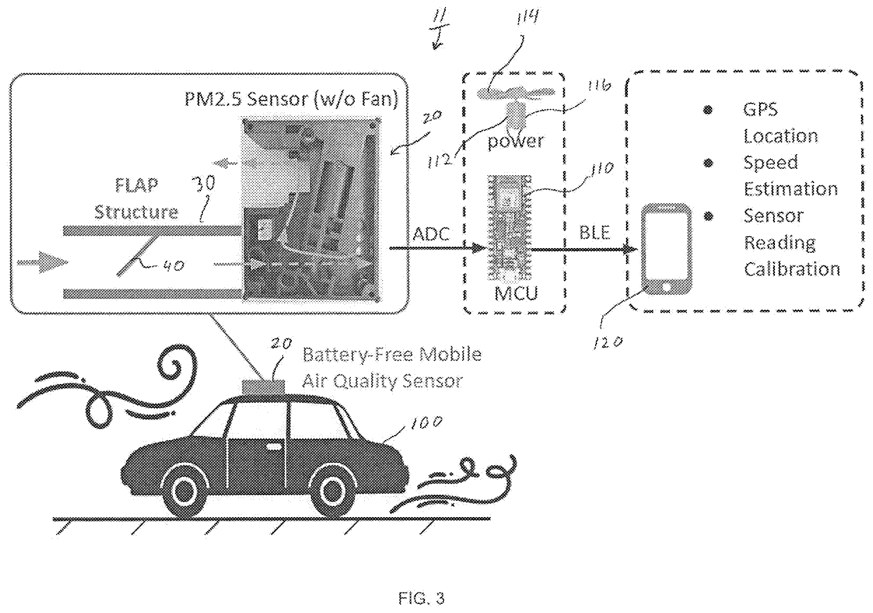

FIG. 2 shows a sensor 20, which is substantially similar to the sensor 10 but does not include the air intake fan 16. The sensor 20 uses a flow adaptive assembly 30 to regulate airflow through the sensor 20. With reference to FIG. 3, a mobile air quality monitoring system 11 includes the sensor 20, which is coupled to a vehicle 100. When the vehicle 100 is moving within a preset range of speeds (e.g., from about 20 mph to about 65 mph, and in embodiments from about 20 mph to about 40 mph), airflow is provided to the sensor 20, which allows the sensor 20 to measure air quality and other properties of the air. The vehicle 100 may be any one of various mobile platforms such as cars, buses, aerial drones, or other vehicles capable of generating airflow for the system's operation.

The sensor 20 is coupled to a microcontroller unit (MCU) 110, which is powered by a turbine 112. The MCU 110 is configured to communicate with a computing device 120. The MCU 110 serves as the central processing hub, receiving data from the sensor 20 and managing system functions. The MCU 110 digitizes the sensor's analog readings using an integrated an analog-to-digital converter (ADC) and transmits the processed data for external analysis. The MCU 110 operates efficiently, powered solely by the turbine 112, enabling energy autonomy.

The computing device 120 may be connected to the MCU 110 via a wireless connection, such as BLUETOOTH® Low Energy (BLE). The computing device 120, such as a smartphone or tablet, receives real-time sensor data and integrates this data with additional information, including GPS location and speed estimation of the vehicle 100, to calibrate and analyze air quality measurements for accurate reporting and mapping. In embodiments, the computing device 120 may be a server configured to communicate with the MCU 110 using any suitable wireless connection, e.g., Wi-Fi, cellular data, etc. The MCU 110 may include storage for data collection and subsequent upload to the server once the MCU 110 can communicate with the computing device 120, e.g., once Wi-Fi or cellular connection is established.

The computing device 120 obtains air quality readings while the vehicle 100 is in motion. As a result, these measurements differ from those obtained by static sensors, requiring calibration to account for spatial and temporal conditions. This is particularly challenging when the vehicle 100 operates at higher speeds (e.g., 20-35 mph). The computing device 120 combines physics-based and data-driven approaches to integrate sensor measurements, vehicle movement data, and ambient environmental information, facilitating the development of a mobility-aware air quality crowdsensing model.

The sensor 10 may consume approximately 0.55 W, which accounts for the combined power usage of the fan 16 and laser module 18. The sensor 20 is operable without use of the fan 16 further reducing power consumption requirements. Additionally, the MCU 110 may use around 0.15 W, e.g., BLE advertisement. The turbine 112 is configured to generate about 1 W of power, which is sufficient to supply energy to a low-power sensing system including the sensor 20 and the MCU 110.

The turbine 112 may be positioned anywhere on the vehicle 100 to harness airflow generated by vehicle's motion, the turbine 112 converts kinetic energy into electrical energy. This energy is used to power the MCU 110 and/or the sensor 20, eliminating the need for external power sources or batteries, thereby enhancing system portability and sustainability.

The turbine 112 includes a rotor 114 having a plurality of blades and a hub. The turbine 112 captures the kinetic energy from the airflow, converting it into rotational energy. The hub connects the blades to a generator 116, which converts the rotational mechanical energy from the rotor 114 into electrical energy. The generator 116 may include a power conversion circuitry and optional power storage to provide uninterrupted power to the MCU 110 and/or the sensor 20.

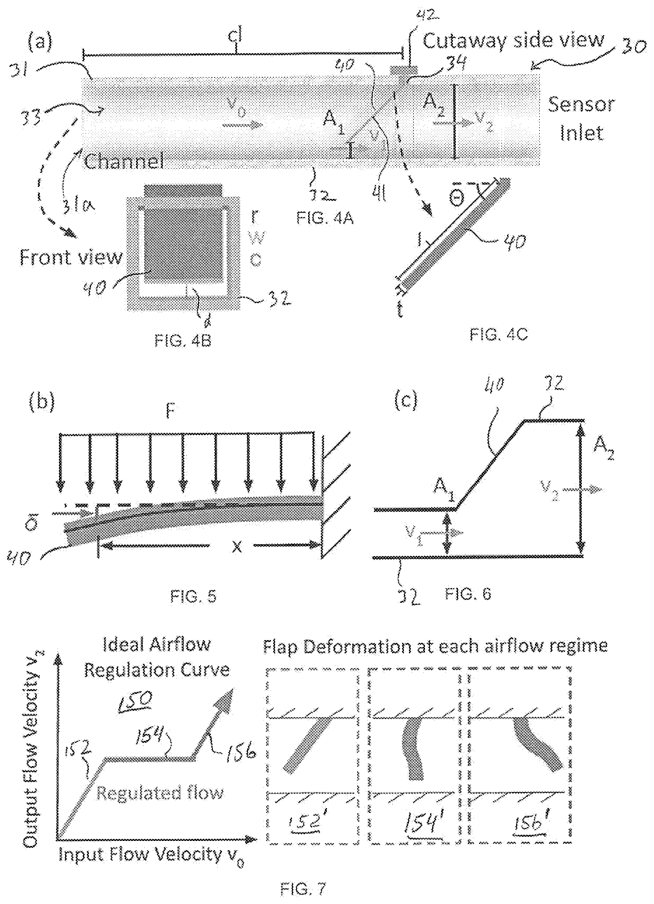

FIGS. 4A-C show the flow adaptive assembly 30, which includes a channel tube 31 that extends from the housing 12 of the sensor 20 and terminates in an intake 31a. The channel tube 31 includes an elastic flap 40, which adapts to the input airflow and provides regulated airflow output to the sensor 20, ensuring consistent measurement conditions under varying external airflow velocities.

The channel tube 31 is designed to extend from the housing 12 of the sensor 20, as depicted in FIGS. 2 and 3. The channel tube 31 has a cross-sectional area matching the dimensions of the inlet opening 17 of the sensor 20, which is shown as a square in FIG. 4B. The channel tube 31 may have any suitable cross-sectional shape, such as circular, oval, rectangular, polygonal, or combinations thereof, depending on the shape of the inlet opening 17. The channel tube 31 is formed from a wall 32 defining channel 33 through the channel tube 31. The channel 33 has a channel length (cl) and a channel cross-sectional dimension (c), which may be a width, diameter, etc. depending on a cross-sectional of the channel tube 31.

When airflow enters the channel 33 within the channel tube 31, the no-slip condition at the channel walls 32 alters the velocity (i.e., flowrate) profile of the airflow. The distance from the channel entrance to the point where the airflow achieves a fully developed profile is referred to herein as an entrance length. The channel tube 31 is designed such that the channel length cl exceeds the entrance length for the airflow. This ensures a stabilized airflow v₀, as illustrated in FIG. 4A, which applies a consistent force on the elastic flap 40, thereby enabling reliable airflow regulation.

With continued reference to FIG. 4A, the elastic flap 40 may include two components: the mounting portion 42 and a flexing portion 41 which may be thinner than mounting portion 42. The elastic flap 40 may be coupled to the channel tube 31 through a slit 34 positioned at the channel length cl from the opening. The slit 34 facilitates the installation of the elastic flap 40 by insertion of the elastic flap 40 through the slit 34 while a mounting portion 42 of the flap 40, which is larger than the slit 34, remains outside the channel 33. The mounting portion 42 seals the slit 34, to prevent air escaping therethrough. In embodiments, the elastic flap 40 may be attached to the inside surface of the walls 32 using any other suitable means, adhesive, ultrasonic welding, etc.

With reference to FIG. 4C, the elastic flap 40 has a thickness (t), length (l), channel angle (0), and width (w). The elastic flap 40 may have a side clearance or relief (r). The bottom of the flap 40 may be spaced apart from the bottom of the channel 33 to provide for minimum cross-sectional area A1 that allows for minimum air flow. The elasticity of the flap 40 is shown in a deformation model of FIG. 5, which is generally represented as a cantilever beam with one fixed end (e.g., along the slit 34). FIG. 6 shows the Venturi effect, which describes flow velocity changes within the channel 33 of varying sizes of cross-sectional areas A1 and A2 due to the deformation, e.g., bending, of the flap 40.

The control of the airflow rate to the sensor 20 leverages the deformation of the elastic flap 40. The deformation produced by the uniform load F on the beam at position x distance from the fixed end can be defined using formula (I):

$$δ = - \frac{{Fx}^{2}}{24 EI} ( 6 {l}^{2} - 4 lx - {x}^{2} ) \qquad ( I )$$

In formula (I), E is the modulus of elasticity, I is the moment of inertia of the cross-section area, and l is the length of the cantilevered flap 40.

When the flap structure is integrated into the channel structure with a tilted angle θ, as shown in FIGS. 4A and 4C, the increase of input airflow v₀ results in a higher load F applied to the flap 40. This will further lead to a larger deformation δ (FIG. 5) and a decreased cross-sectional area A₁ (FIG. 6), which allows the flow to pass through. This change of A₁ controls the bypassing flow rate and regulates the output flow rate described by the Venturi effect, as shown in FIG. 6 where the airflow is bounded by the walls 32 of the channel 33 and further by the flap 40 being fully deformed. Considering the cross-sectional area A and flow velocity v at and after the elastic flat (A₁, v₁ and A₂, v₂), Venturi effect describes their relationship as A₁×v₁=A₂×v₂. Since A₂ is a constant, v₂ can only be regulated when A₁×v₁ remains a constant. This is achieved because A₁=ƒ(δ) and v₁ change simultaneously when velocity v₀ of the input airflow to the channel 33 changes. When v₀ increases, A₁ decreases due to the deformation δ, while v₁ increases due to a larger input flow rate.

Based on formula (I), the deformation δ is directly proportional to the length l of elastic flap 40. Thus, when the same uniform force is applied to flap 40 having a longer length, this will result in a larger δ. The δ is inversely proportional to the moment of inertia I, which is a function of flap thickness t and width w. Accordingly, the increase in thickness t and width w will increase the moment of inertia I, making the flap 40 more rigid and, hence, difficult to deform.

The flap 40 may have a thickness from about 0.1 mm to about 2 mm, in embodiments from about 0.3 mm to about 1 mm. The length of the flap 40 depends on the dimensions of the channel 33 and a suitable measure of the length of the flap 40 may be the bottom clearance (e.g., distance (d)) that provides for the minimum area A₁. In embodiments, the distance (d) between the bottom of the flap 40 and the channel 33 may be the same as the relief (r) or larger. In embodiments, the relief (r) may be from about 0.5 mm to about 2 mm and the bottom distance (d) may be from about 0.5 mm to about 5 mm, and in embodiments, the bottom distance (d) may be from about 1 mm to about 3 mm.

The elastic flap 40 can be fabricated using a variety of manufacturing techniques tailored to achieve the desired mechanical properties, dimensions, and structural integrity. The elastic flap 40 may be formed using additive processing techniques such as binder jetting, fused deposition modeling, fused filament fabrication, stereolithography, vat photopolymerization, and the like. In embodiments, the elastic flap 40 may be formed using molding techniques, such as injection molding, compression molding, transfer molding, and the like. In furthermore, additional manufacturing techniques may also be used, such as laser cutting, layer bonding, extrusion, casting, vacuum forming, and the like. Suitable materials for making the elastic flap 40 include any elastomer or combinations thereof, such as silicone rubber, natural rubber, polyurethanes, thermoplastic elastomers, nitrile rubber, photopolymer resins, such as RESIONE® F80, and the like.

The flap 40 is configured to morph and regulate the output airspeed 12 to the inlet 17 of the sensor 20 within a range of input airspeed v₀, which is considered as the operational range for the sensing operation. FIG. 7 shows an idealized airflow regulation curve 150. When the external flow velocity is lower than a threshold for deforming or tilting the flap 40, the force exerted on the flap 40 by airflow is not sufficient to produce any deformation. When the external flow velocity v₀ increases in this range, the output flow velocity 12 increases proportionally, shown as a line 152 in FIG. 7 and a corresponding schematic diagram 152′ of the flap 40. With the external flow-induced force exceeding the critical load limit for the flap 40, the flap deformation δ changes with the flow velocity. The deformation δ determines the cross-sectional area A₁, which achieves the minimum value when δ≈l cos θ. In this range of external flow velocity, the output flow velocity can be regulated due to the Venturi effect, which is marked as a line 154 in FIG. 7 a corresponding schematic diagram 154′ of the flap 40. When v₀ further increases, the flap deformation δ exceeds the threshold to decrease the cross-section area A₁. Instead, A₁ increases as shown in a corresponding schematic diagram 156′ of the flap 40. In this stage, both A₁ and v₁ increase with v₀; therefore, v₂ increases, as represented by a line 156. The flap 40 is configured so that the v₀ of the regulated stage aligns with the designated driving speed range, which may be from about 20 mph to about 65 mph, and in embodiments from about 20 mph to about 40 mph.

The initial angle θ at which the flap 40 is disposed relative to the wall 32 of the tube 31 affects the dynamic control of the airflow by the flap 40. In particular, the angle of the flap 40 toward the direction of the airflow may be from about 30° to about 60°, and in embodiments from about 35° to about 55°. The flap 40 is sufficiently rigid such that the flap 40 maintains this angle while the airflow is below the preset threshold as described above with respect to the line 152 of the regulation curve 150. Airflow regulation occurs in the line 154 of the curve 150 once airflow velocity is sufficient to deform the flap 40 which bends the flap, increasing the angle θ and/or deforming the flap 40 (see diagram 154′). The length of the flap 40 also affects flow regulation. The length l cannot be too large that during deformation the flap 40 contacts the wall 32 of the tube 31. In addition, the length is selected to provide for sufficient input airflow at low speeds that do not deform the flap 40.

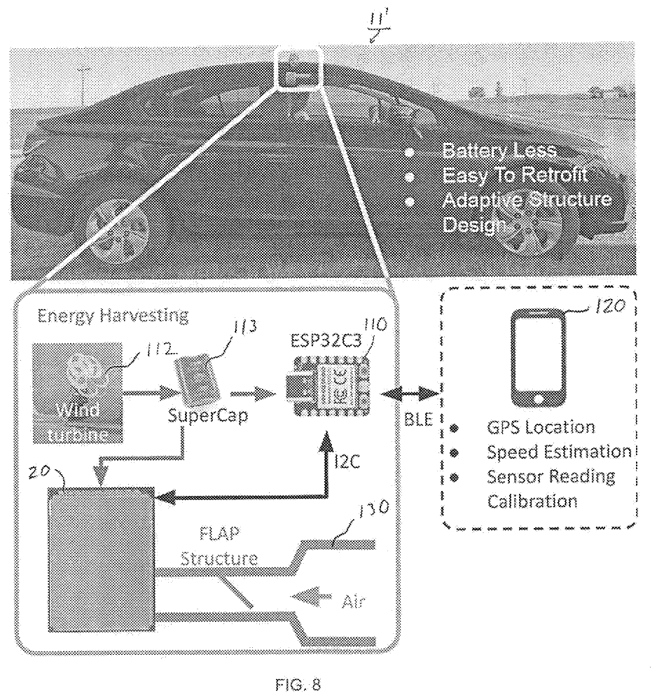

FIGS. 8 and 9 show another embodiments of a mobile air quality monitoring system 11′, which includes the sensor 20 coupled to a vehicle 100. The system 11′ is substantially similar to the system 11 of FIGS. 2 and 3 and includes similar components with different components and functionality described in further detail below. The system 11′ also uses the sensor 20 but incorporates a different flow adaptive assembly 130 to regulate airflow through the sensor 20.

The system 11′ includes the MCU 110, such as a ESP32-C3 from Espressif Systems of Shanghai, China, the turbine 112, and the computing device 120. The turbine 112 may be coupled to a power conversion circuitry and optional power storage or a supercapacitor 113 to provide uninterrupted power to the MCU 110 and/or the sensor 20.

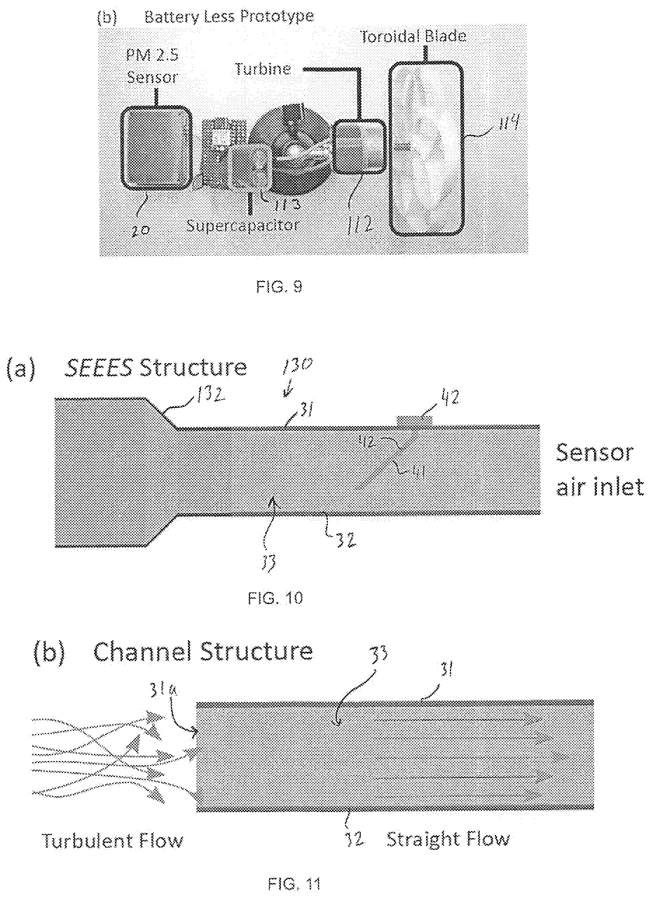

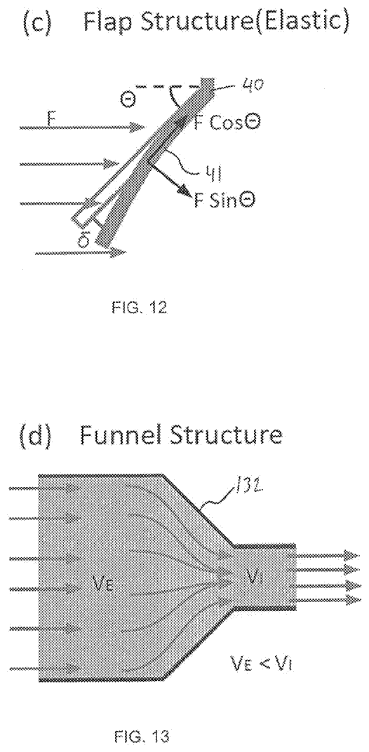

FIGS. 10-13 show the flow adaptive assembly 130, which is similar to the flow adaptive assembly 30 of FIGS. 4A-C, 5, and 6. Similar to the assembly 30, the flow adaptive assembly 130 includes the channel tube having the wall 32 and defining channel 33 through the channel tube 31. The flow adaptive assembly 130 also includes the elastic flap 40 having the mounting portion 42 and the flexing portion 41 which may be thinner than mounting portion 42.

When the vehicle is in motion, as shown in FIG. 11, external turbulent airflow enters a channel structure, where friction between the airflow and the channel walls gradually stabilizes the flow. As with fluid entering a pipe, the combination of viscosity and wall friction causes the initially disordered flow to become more laminar over a defined entrance length. This entrance length is related to the Reynolds number and the channel diameter (L∝Re·D), where the Reynolds number is itself proportional to the flow velocity. Accordingly, the channel length may be selected to accommodate a range of expected airflow velocities and to promote flow stabilization prior to reaching downstream components. The wall-induced friction also contributes to straightening the flow, particularly near the boundaries of the channel cross-section.

With reference to FIGS. 10 and 13, The flow adaptive assembly 130 also includes a funnel structure 132 attached to the intake 31a of the channel tube 31. The funnel structure 132 is configured to condition and accelerate the external airflow entering the channel 33 to improve flow regulation across varying vehicle speeds. In particular, the funnel structure 132 includes an inlet having a first cross-sectional area A_{E}, a transition region, and an outlet having a second, smaller cross-sectional area A_{I} that is substantially equal to the cross-sectional area of the channel 33. As airflow transitions from the larger inlet area to the narrower outlet area, the velocity of the airflow increases while the static pressure decreases, in accordance with the Bernoulli principle. This relationship is expressed as A_{E}·v_{E}=A_{I}·v_{I}, where v_{E} and v_{I} are the respective air velocities at the inlet and outlet.

The resulting increase in air velocity at the outlet enhances the force exerted on the flexing portion 41 of the elastic flap 40, thereby modifying the flap's deformation response and shifting the operating range of the flow regulation mechanism. The transitional region of the funnel structure 132 is shaped to provide a gradual reduction in cross-sectional area, which helps to prevent adverse flow effects such as backpressure or turbulence that may result from abrupt changes in flow path geometry. The combination of the funnel structure 132 and the elastic flap 40 allows the flow adaptive assembly 130 to achieve more consistent and tunable regulation of airflow into the air quality sensor during varying vehicular motion conditions.

From the foregoing, it will be appreciated that specific embodiments of the disclosure have been described herein for purposes of illustration, but that various modifications may be made without deviating from the scope of the disclosure. Accordingly, the disclosure is not limited except as by the appended claims. The following Examples illustrate embodiments of the present disclosure. These Examples are intended to be illustrative only and are not intended to limit the scope of the present disclosure. Also, parts and percentages are by weight unless otherwise indicated.

### EXAMPLES

### Example 1

This example describes computer simulation of the flow adaptive assembly according to the present disclosure.

ANSYS™ physics simulation software was used to model the fluid-structure interaction of airflow through the channel and the deformation of elastic flap. For simplification, it was assumed that airflow in simulation is unidirectional. The channel tube length cl was set as a constant, which empirically shows effectiveness in straightening the input flow.

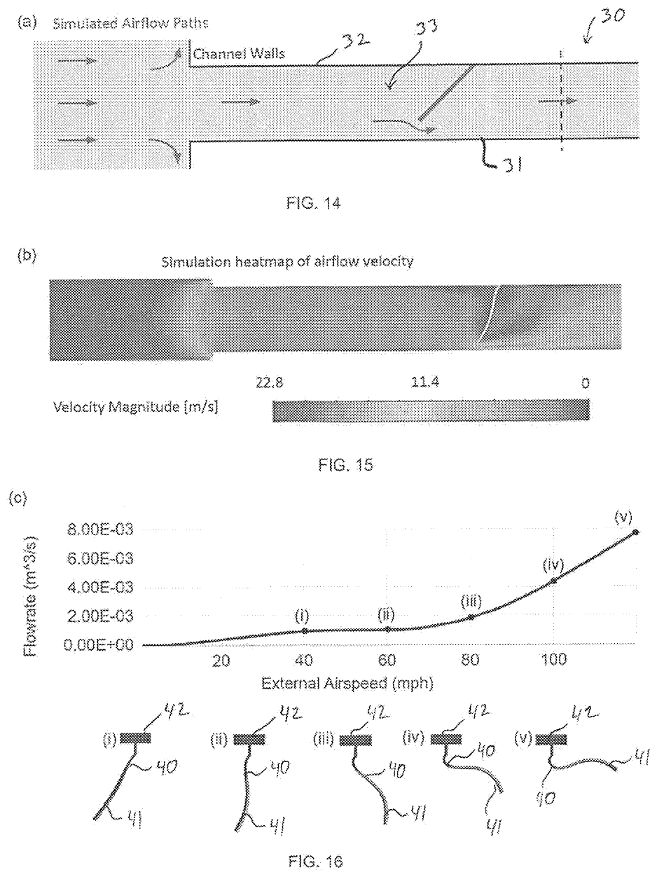

FIG. 14 shows the simulation setup for the channel tube and the elastic flap. The material profile of the elastic flap was created using the material properties of RESIONE® F80 resin and natural rubber. The arrows show the directions of the airflow in and outside the channel tube. The simulation measured the air volume beyond the dotted vertical line, which represents the inlet of the air quality sensor. The simulation was performed with the following flap dimensional parameters as follows: t=0.4 mm, θ=45, and l=15 mm.

FIG. 15 shows the heatmap of the air velocity at different sections in the channel tube. It was observed that the channel tube regulates the airflow velocity after the channel entrance (with a short entrance length) and before the elastic flap. When the external airflow velocity is set to 22.8 m/s, the input airflow velocity v0 is around 5 m/s, which deforms the elastic flap. The reduced cross-sectional area A₁ at the flap increases the flow velocity, and the Venturi effect results in a reduced flow rate after the flap.

FIG. 16 shows the simulated flap shape and measurements of airflow over different external airflow velocities. When the external airflow velocity reaches 40 mph, the flap deformation was observed, as shown in (i), and a regulated flow velocity was observed after this point. The flap deformation at 60 mph, as shown in (ii), depicts a larger deformation δ than that of 40 mph. The regulation curve 150 of FIG. 7 shows that the output airspeed was regulated in the range of 45 to 65 mph external airflow velocities. For the airflow velocity higher than 65 mph, as shown in (iii), (iv), and (v), the deformation δ exceeded the threshold resulting in minimum A₁, and the structure can no longer regulate the flow in this range. This observation matches the analysis of the ideal airflow regulation curve.

### Example 2

This example describes airflow velocity measurements on a vehicle for use with the flow adaptive assembly according to the present disclosure.

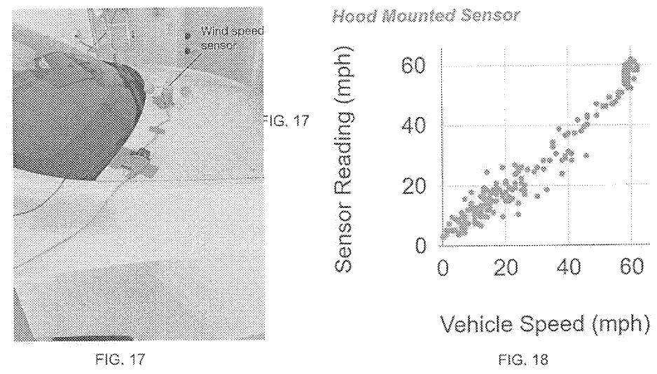

To characterize the external airspeed when mounted on the vehicle, experiments were conducted to measure the airspeed near the vehicle's surface when driving. FIG. 17 depicts the experiment setup, where a wind speed sensor was mounted on the hood of the car. Vehicle speed was acquired by recording a video of the vehicle odometer. On the day of testing, winds of around 7 miles per hour with 11 miles per hour gusts were also recorded. FIG. 18 shows the results for the observed airflow velocity with the change in vehicle speed. It was observed that there is a linear trend between the airflow speed and the vehicle speed, with some deviations among data points. This deviation was caused by the wind in real-world scenarios.

### Example 3

This example describes characterization of the flow adaptive assembly according to the present disclosure.

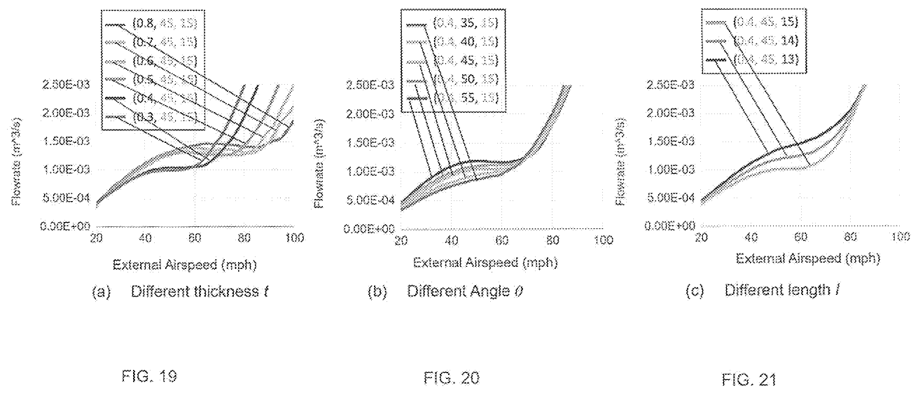

To better understand the flap's response to different external airflow velocities, simulations were also conducted with varying parameters, including flap thickness t, angle θ, and flap length l. FIGS. 19-21 show the simulation results for airflow flow rate through the channel tube versus the external airspeed at varying parameters.

When thickness t increases, the deformability of the structure decreases, and therefore, the force needed to deform the elastic flap increases. As a result, the regulated airspeed ranges at higher airspeeds. FIG. 19 depicts the regulation curve when thickness t increases from 0.4 to 0.8 mm. It was observed that for t=0.3 mm, the regulation curve starts to flat around 40 mph. When t increases to 0.8 mm, this value increases to 60 mph.

The structure of 0.4 mm showed the longest flat region on its regulation curve. Therefore, other parameters (angle and length) were evaluated with thickness t=0.4 mm. When angle θ of the flap relative to the tube increases, the airflow applies increased force to the elastic flap thereby deforming it. Therefore, identifying the parameter θ is useful for achieving the line 154 portion of the regulation. The angle θ was investigated ranging from 35° to 55° and their respective regulation curves were plotted in FIG. 20. When θ increases to 55°, there is a decrease in airflow before the elastic flap fully deforms providing for a larger cross-sectional area and flow increases.

The length of the flap I also impacts the vertical force applied thereto. Therefore, different values of l from 13 mm to 15 mm were also investigated. FIG. 21 shows the simulation outcomes of different l values, where the shorter the length is, the less effective the regulation is. When l reaches 15 mm, which provides for smallest opening, the flap almost reached the channel bottom when deformed.

### Example 4

This example describes the power consumption characteristics and energy harvesting capabilities of the mobile air quality monitoring system with the flow adaptive assembly according to the present disclosure.

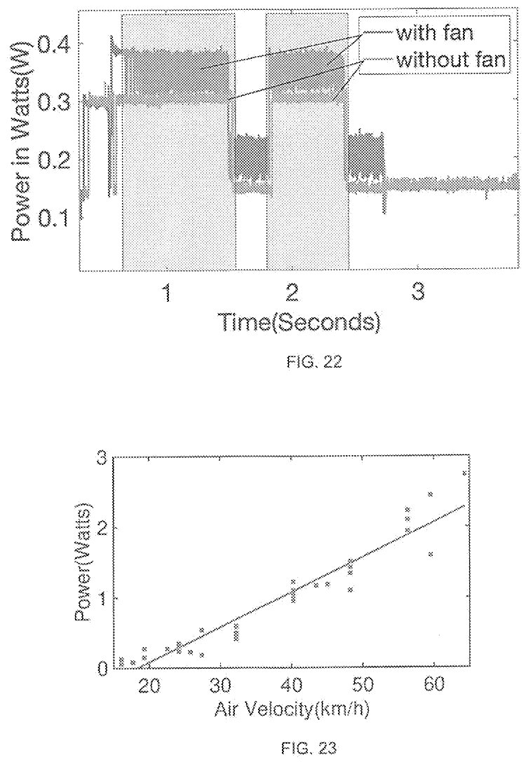

FIG. 22 illustrates the power consumption profile of various sensing components in a prototype implementation of the mobile air quality monitoring system. To measure the instantaneous power usage, a 4Ω shunt resistor was connected in series with the circuit. The measured components included a microcontroller, a PM2.5 sensor with an integrated laser source, and a sensor fan.

The power consumption of the microcontroller alone was measured to be approximately 0.144 W when the sensor was idle. Activation of the laser source increased the power draw to approximately 0.3 W, and when both the laser and fan were active, the total power consumption averaged approximately 0.36 W. FIG. 22 includes a bottom trace representing operation without the sensor fan, and a top trace representing operation with the fan enabled. The sensor was active between 0 and 0.5 seconds and deactivated after 2.7 seconds. The laser source was enabled in two intervals: from 0.6 to 1.5 seconds and again from 1.8 to 2.4 seconds, corresponding to the elevated consumption regions highlighted in yellow. Based on these measurements, the relative power contributions of the microcontroller, laser, and fan were approximately in the ratio 3:3:1. Accordingly, elimination of the fan through the use of a passive airflow control mechanism, such as the elastic flap structure described herein, yields a reduction in sensor energy consumption of approximately 25%.

FIG. 23 shows the power generation performance of the mobile air quality monitoring system when configured with a wind turbine for battery-free operation. To evaluate the energy harvesting capability, the prototype was mounted on a moving vehicle, and power output was measured at various vehicle speeds ranging from 16 km/h to 72 km/h. Wind velocity was recorded using an external anemometer, and corresponding power output was plotted, with measured data points shown in orange and a fitted curve shown in blue.

These results demonstrate that the wind turbine is capable of generating sufficient power to support PM2.5 sensing during typical vehicle operation in residential and urban environments, where speed limits generally range from 40 km/h (25 mph) to 56 km/h (35 mph). Thus, the flow adaptive assembly enables effective, battery-free mobile air quality monitoring by leveraging vehicle-induced airflow for both sensor operation and flow regulation.

### Example 5

This example describes sensor airflow input control according to the present disclosure.

Simulations were performed using the ANSYS™ physics simulator to evaluate the performance of the adaptive airflow regulation structures of the flow adaptive assembly. The simulations modeled fluid-structure interactions within the airflow pathway and assumed laminar flow conditions at the inlet to simplify analysis. The airflow velocity was varied across a range of values to measure the corresponding airflow rate at the sensor inlet and the degree of deformation of the elastic flap structure.

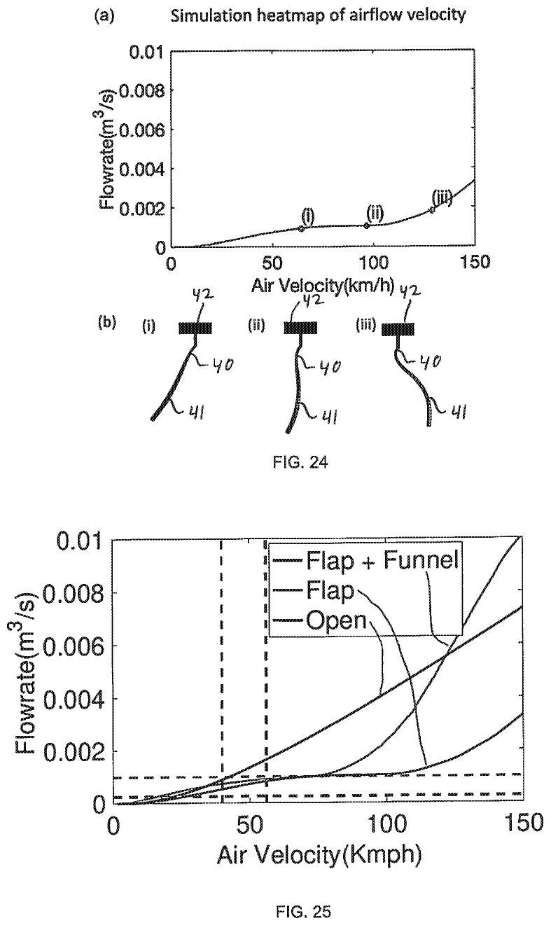

FIG. 24 illustrates the simulation setup and results. A top plot of FIG. 24 depicts the output flow rate measured at the sensor inlet for various external air velocities after passing through the elastic flap. FIG. 24 also shows the simulated deformation of the elastic flap at three representative external velocities: 64.4 km/h, 96.6 km/h, and 128.7 km/h. The corresponding flow rates at these velocities were measured to be approximately 0.935×10⁻³, 1.054×10⁻³, and 1.841× 10⁻³ m³/s, respectively. The deformation at lower velocity (64.4 km/h) was minimal, resulting in a nearly linear increase in flow rate. However, between 64.4 km/h and 96.6 km/h, the deformation of the flap caused the increase in flow rate to plateau, with a change of only 0.119×10⁻³ m³/s despite a 32.2 km/h increase in velocity. This behavior indicates that the elastic flap structure effectively regulates the flow rate over a defined range of external air velocities by adjusting its geometry in response to airflow pressure.

While the elastic flap structure alone provides effective regulation in the 64-96 km/h range, it does not fully align with the desired operational range of approximately 40-56 km/h, which corresponds to typical vehicle speeds in residential and urban environments. To address this, the simulation further evaluated the addition of the funnel structure described earlier. FIG. 25 presents a comparative analysis of output flow rate across three structural configurations: an open channel (without flap or funnel), a channel with elastic flap, and a channel with both elastic flap and funnel.

As shown in FIG. 25, the open channel configuration resulted in a nearly linear increase in flow rate, with a relatively narrow window (24-42 km/h) in which the output fell within the operational range of mini fans (0.25×10⁻³ to 0.99×10⁻³ m³/s). The configuration with the flap structure extended the usable range to 40-56 km/h, producing flow rates between approximately 0.53×10⁻³ and 0.83×10⁻³ m³/s. The combination of flap and funnel structures further stabilized this output, yielding a narrower and flatter regulation curve with flow rates between approximately 0.74×10⁻³ and 0.90×10⁻³ m³/s over the same velocity range. These results confirm that the flow adaptive assembly, when equipped with both funnel and flap structures, is well suited for regulating airflow in battery-less mobile sensing applications over common vehicular speed ranges.

Alternate embodiments may be devised without departing from the spirit or the scope of the present technology. Additionally, well-known elements of embodiments of the systems, apparatuses, and methods have not been described in detail or have been omitted so as not to obscure the relevant details of the systems, apparatuses, and methods.

The terminology used herein is for the purpose of describing particular embodiments only and is not intended to be limiting. The terms “comprises,” “comprising,” or any other variation thereof are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element. The terms “including” and/or “having,” as used herein, are defined as comprising (i.e., open language). The terms “a” or “an”, as used herein, are defined as one or more than one. The term “plurality,” as used herein, is defined as two or more than two. The term “another,” as used herein, is defined as at least a second or more. The description may use the terms “embodiment” or “embodiments,” which may each refer to one or more of the same or different embodiments.

When the terms “coupled” and “connected,” along with their derivatives, are used, these terms are not intended as synonyms for each other. For example, “connected” may be used to indicate that two or more elements are in direct physical or electrical contact with each other. “Coupled” may mean that two or more elements are in direct physical or electrical contact (e.g., directly coupled) or that two or more elements are not in direct contact with each other but yet still cooperate or interact with each other (e.g., indirectly coupled).

For the purposes of the description, a phrase in the form “A/B” or in the form “A and/or B” or in the form “at least one of A and B” means (A), (B), or (A and B), where A and B are variables indicating a particular object or attribute. When used, this phrase is intended to and is hereby defined as a choice of A or B or both A and B, which is similar to the phrase “and/or”. Where more than two variables are present in such a phrase, this phrase is hereby defined as including only one of the variables, any one of the variables, any combination of any of the variables, and all of the variables, for example, a phrase in the form “at least one of A, B, and C” means (A), (B), (C), (A and B), (A and C), (B and C), or (A, B and C).

Relational terms such as first and second, top and bottom, and the like may be used solely to distinguish one entity or action from another entity or action without necessarily requiring or implying any actual such relationship or order between such entities or actions. The description may use perspective-based descriptions such as up/down, back/front, top/bottom, and proximal/distal. Such descriptions are merely used to facilitate the discussion and are not intended to restrict the application of disclosed embodiments. Various operations may be described as multiple discrete operations in turn, in a manner that may be helpful in understanding embodiments; however, the order of description should not be construed to imply that these operations are order dependent.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. As used herein, the terms “substantial” and “substantially” means, when comparing various parts to one another, that the parts being compared are equal to or are so close enough in dimension that one skill in the art would consider the same. Substantial and substantially, as used herein, are not limited to a single dimension and specifically include a range of values for those parts being compared. The range of values, both above and below (e.g., “+/−” or greater/lesser or larger/smaller), includes a variance that one skilled in the art would know to be a reasonable tolerance for the parts mentioned.

Various embodiments of the systems, apparatuses, and methods have been described, and in many of the different embodiments many features are similar. To avoid redundancy, repetitive description of these similar features may not be made in some circumstances. It shall be understood, however, that description of a first-appearing feature applies to the later described similar feature and each respective description, therefore, is to be incorporated therein without such repetition.

What is claimed is:

1. A system for mobile air quality monitoring, comprising:
an air quality sensor including a housing and an inlet;
a flow adaptive assembly coupled to the inlet of the air quality sensor, the flow adaptive assembly including:
a channel tube extending from the housing, the channel tube having an intake and a length sufficient to provide a stabilized airflow entering the inlet of the air quality sensor; and
an elastic flap positioned within the channel tube, wherein the elastic flap is configured to deform in response to variations in the airflow entering the channel tube, thereby regulating flowrate of the stabilized airflow into the inlet of the air quality sensor; and
a microcontroller unit operatively connected to the air quality sensor, the microcontroller unit configured to process data generated by the air quality sensor.

2. The system according to claim 1, wherein the flow adaptive assembly further includes a funnel coupled to an intake of the channel tube.

3. The system according to claim 1, wherein the air quality sensor is configured to be mounted on a vehicle.

4. The system according to claim 3, further comprising:
a turbine coupled to the microcontroller unit, the turbine configured to be mounted on the vehicle, to harvest kinetic energy from the airflow, and to generate electrical energy to power the microcontroller unit and the air quality sensor.

5. The system according to claim 3, further comprising:
a computing device operatively connected to the microcontroller unit via a wireless communication interface, the computing device configured to receive data from the microcontroller unit, integrate the data with location and speed information of the vehicle, and calibrate air quality measurements from the air quality sensor.

6. The system according to claim 1, wherein the air quality sensor is configured to measure particulate matter concentration in the air.

7. The system according to claim 1, wherein the elastic flap includes a mounting portion for securing the flap within the channel tube and a flexing portion configured to deform under airflow pressure.

8. The system according to claim 1, wherein the channel tube has a circular, rectangular, oval, or polygonal cross-sectional geometry.

9. The system according to claim 1, wherein the elastic flap is configured to deform within a pre-defined range of airflow velocities.

10. The system according to claim 1, wherein the elastic flap is disposed at an initial angle relative to a wall of the channel tube, and the elastic flap is configured to maintain the initial angle until the airflow exceeds a threshold velocity.

11. The system according to claim 10, wherein the initial angle is 30° to 60°.

12. An air quality sensor comprising:
a housing including a sensor component for measuring air quality;
an inlet;
a flow adaptive assembly coupled to the inlet, the flow adaptive assembly including:
a channel tube extending from the housing, the channel tube having a length sufficient to provide a stabilized airflow entering the inlet; and
an elastic flap positioned within the channel tube, wherein the elastic flap is configured to deform in response to variations in the airflow entering the channel tube, thereby regulating flowrate of the stabilized airflow into the inlet.

13. The air quality sensor according to claim 12, wherein the flow adaptive assembly further includes a funnel coupled to an intake of the channel tube.

14. The air quality sensor according to claim 12, wherein the elastic flap includes a mounting portion for securing the flap within the channel tube and a flexing portion configured to deform under airflow pressure.

15. The air quality sensor according to claim 12, wherein the channel tube has a circular, rectangular, oval, or polygonal cross-sectional geometry.

16. The air quality sensor according to claim 12, wherein the elastic flap is configured to deform in response to being exposed to a pre-defined range of airflow velocities.

17. The air quality sensor according to claim 12, wherein the elastic flap is disposed at an initial angle of from about 30° to 60° relative to a wall of the channel tube, and the elastic flap is configured to maintain the initial angle until the airflow entering the channel tube exceeds a threshold velocity.

18. The air quality sensor according to claim 12, wherein the sensor component is configured to measure particulate matter concentration in the air.

19. The air quality sensor according to claim 18, wherein the sensor component includes a laser module and a photodetector for measuring concentration of the particulate matter in the airflow, wherein particulate matter is at least one of PM1.0, PM2.5, or PM10.

20. The air quality sensor according to claim 12, wherein the sensor component includes a gas sensor configured to measure concentration or detect at least one of carbon monoxide, carbon dioxide, nitrogen dioxide, sulfur dioxide, ozone, a volatile organic compound, hydrogen sulfide, ammonia, or radon.
